(19) United States
(12) Patent Application Publication
(10) Pub. No.: US 2023/0003679 A1
(43) Pub. Date: Jan. 5, 2023

(54) AMPEROMETRIC SENSOR FOR MEASURING FREE CHLORINE WITH REFERENCE ELECTRODE HAVING A GOLD ELECTRODE SURFACE COMPOSED OF A STRING OF ELECTRICALLY CONNECTED, SPACED APART SURFACE PARTS

(71) Applicant: INNOVATIVE WATER CARE, LLC, Alpharetta, GA (US)

(72) Inventors: Maurice Paul Tax, Joure (NL); Jerome Logie, Sainte-Euphemie (FR); Matthew Gilbert Muller, Cleveland, TN (US)

(73) Assignee: INNOVATIVE WATER CARE, LLC, Alpharetta, GA (US)

(21) Appl. No.: 17/756,947

(22) PCT Filed: Dec. 6, 2019
(86) PCT No.: PCT/EP2019/084039

§ 371 Date: Jun. 6, 2022

(51) Int. Cl. G01N 27/30; G01N 33/18; G01N 27/416

(52) U.S. Cl. CPC G01N 27/301; G01N 33/182; G01N 27/4168

(57) ABSTRACT

The present disclosure relates to an amperometric sensor for measuring free chlorine, which sensor comprises:

an elongate body with a tip, wherein the circumferential surface of the body constitutes a counter electrode;

a reference electrode having a silver/silverchloride electrode surface arranged on the tip of the elongate body; and

a working electrode having a gold electrode surface arranged on the tip of the elongate body wherein the gold electrode surface is composed out of a string of electrically connected, spaced apart surface parts.

### CROSS-REFERENCE TO RELATED APPLICATIONS

This is a National Stage of International Application No. PCT/EP2019/084039, filed Dec. 6, 2019.

### TECHNICAL FIELD

The present disclosure relates to an amperometric sensor for measuring free chlorine, which sensor comprises:

an elongate body with a tip, wherein the circumferential surface of the body constitutes a counter electrode;

a reference electrode having a gold electrode surface arranged on the tip of the elongate body; and

a working electrode having a silver/silverchloride electrode surface arranged on the tip of the elongate body.

Such an amperometric sensor is used to detect ions in a solution based on electric current or changes in electric current. To this end, the potential of the working electrode is maintained at a constant level with respect to the reference electrode by adjusting the current at the counter electrode. This is typically achieved by connecting the amperometric sensor to a potentiostat.

### BACKGROUND

In order to measure free chlorine, it is known, from for example US 20090014329, to maintain a potential of 0.2 V between the working electrode and the reference electrode. By measuring the current flowing through the working electrode a reliable indication for the amount of free chlorine is obtained.

Due to the formation of deposits onto the counter and reference electrodes the sensitivity typically decreases over time. This can be slowed by periodically reversing the current, such that the deposits are loosened from the electrode surfaces. Another, complimentary option is to increase the surfaces of the electrodes. However, especially for the gold electrode surface of the reference electrode, this would be costly. Furthermore, even with the technique of reversing the current to clean the electrode surfaces, especially the gold electrode surface will still deteriorate gradually, such that the quality of the measurements will deteriorate over time.

### BRIEF SUMMARY

Amperometric sensors for measuring free chlorine, and a combination of the amperometric sensor and a potentiostat are provided herein. In an embodiment, an amperometric sensor includes an elongate body with a tip, wherein a circumferential surface of the body constitutes a counter electrode. The amperometric sensor further includes a reference electrode having a gold electrode surface arranged on the tip of the elongate body. The amperometric sensor further includes a working electrode having a silver/silverchloride electrode surface arranged on the tip of the elongate body. The gold electrode surface is composed out of a string of electrically connected, spaced apart surface parts.

In another embodiment, a combination of an amperometric sensor, as described above, and a potentiostat is provided. The potentiostat is connected to the counter electrode, the working electrode and the reference electrode for maintaining the potential of the working electrode at a constant level with respect to the reference electrode by adjusting the current at the counter electrode.

### BRIEF DESCRIPTION OF THE DRAWINGS

The present disclosure will hereinafter be described in conjunction with the following drawing figures, wherein like numerals denote like elements, and wherein:

FIG. 1 shows a schematic perspective view of an amperometric sensor according to an embodiment;

FIG. 2 shows a schematic diagram of a combination according to an embodiment with an amperometric sensor of FIG. 1; and

FIG. 3 shows a diagram of the influence of cyanuric acid on the sensitivity of an embodiment of the amperometric sensor according to an embodiment.

### DETAILED DESCRIPTION

The following detailed description is merely exemplary in nature and is not intended to limit the present disclosure or the application and uses thereof. Furthermore, there is no intention to be bound by any theory presented in the preceding background or the following detailed description.

It is desired to provide an amperometric sensor, which can be used for a prolonged time, without substantial deterioration of the sensitivity.

This object is achieved with an amperometric sensor as contemplated herein, which is exemplified in that the gold electrode surface is composed out of a string of electrically connected, spaced apart surface parts.

When the amperometric sensor as contemplated herein is used for measuring free chlorine, a potential of 0.2 V is provided over the reference electrode and the working electrode. By applying a different potential, the amperometric sensor could also be used for measuring other ions, such as bromine. The potential decreases over the string of electrically connected, spaced apart surface parts of the gold electrode surface, such that basically only the first surface part of the electrode is used to contribute to the measurements of the amperometric sensor as contemplated herein. Only when the first surface part is deteriorated to a certain degree, the next surface part in the string will contribute to the measurements of the amperometric sensor. This allows for maintaining a good sensitivity of the sensor over a prolonged time, as there will still be a virtually new surface part be available for the measurements.

An additional advantage found by the applicant is that the amperiometric sensor hardly influenced by the concentration of other chemicals, in particular cyanuric acid.

In a preferred embodiment of the amperometric sensor as contemplated herein the surface parts are identically shaped and dimensioned, preferably rectangular. Having identically shaped and dimensioned surface parts ensures that no substantial variations in the measurements occur, when the effective electrode surface shifts from a first surface part to the next surface part in the string.

In a further preferred embodiment of the amperometric sensor as contemplated herein the gold electrode surface is provided as a layer arranged on a substrate by wafer production techniques, such as lithographic production techniques.

By providing the gold electrode surface as a layer arranged on a substrate by wafer production techniques, i.e. on a chip, the production costs can be kept low as well as the material costs can be kept low. Using the wafer production techniques a small gold electrode surface can be provided with the required string of electrically connected, spaced apart surface parts.

An additional advantage is that by arranging the gold electrode surface on a chip, the chip can easily be mounted in the amperometric sensor and could also be exchanged easily, when the gold surface of all surface parts has deteriorated too much.

Preferably, the counter electrode has a titanium electrode surface. Typically, stainless steel would be chosen for the counter electrode, as this is a low cost material, which allows for a large electrode surface. However, using the amperiometric sensor to measure free chlorine, will still result in that the stainless steel dissolves over time. By providing the counter electrode with a titanium electrode surface, this disadvantage is resolved and as result, the potentiostat connected to such an amperometric sensor as contemplated herein will need less calibration.

In a further embodiment of the amperometric sensor as contemplated herein the reference electrode is composed out of a sintered Ag/AgCl powder.

The present disclosure further relates to a combination of an amperometric sensor as contemplated herein and a potentiostat connected to counter electrode, working electrode and reference electrode for maintaining the potential of the working electrode at a constant level with respect to the reference electrode by adjusting the current at the counter electrode.

With the potentiostat it is ensured that the potential between the working electrode and reference electrode is constant, for measuring free chlorine typically 0.2 V. This allows for the current to be measured at the working electrode and to derive from the measured current a reference for the amount of free ions, such as free chlorine, in a solution.

In a preferred embodiment of the combination as contemplated herein the potentiostat comprises a control feature for maintaining the potential of the working electrode at a constant level with respect to the reference electrode, which control feature has a compensating feature, which measure the resistance between electrode surfaces of at least the working electrode and the reference electrode and compensate for the measured resistance.

Measuring the resistance between the working electrode and reference electrode provides an indication for the conductivity of the solution, in which the free ions, such as free chlorine or bromine, is measured. This conductivity has a small influence on the accuracy of the measurements. By measuring the resistance, the potentiostat can compensate for this influence, such that the accuracy is further increased.

Although measuring the resistance between the working electrode and reference electrode is preferred, it is also contemplated herein to provide a separate resistance sensor, typically embodied as two separate electrodes.

In yet another embodiment as contemplated herein the potentiostat further comprises a signal generator for adjusting the constant level according to a predefined signal pattern.

By varying the constant level according a predefined signal pattern, the electrodes can be cleaned from any deposits, by reversing the current, but also different ions could be detected simultaneously, by measuring the current at the correct constant level. This allows for chlorine to be measured at the constant level of 0.2 V, while bromine can be measured at the constant level of 0.1 V.

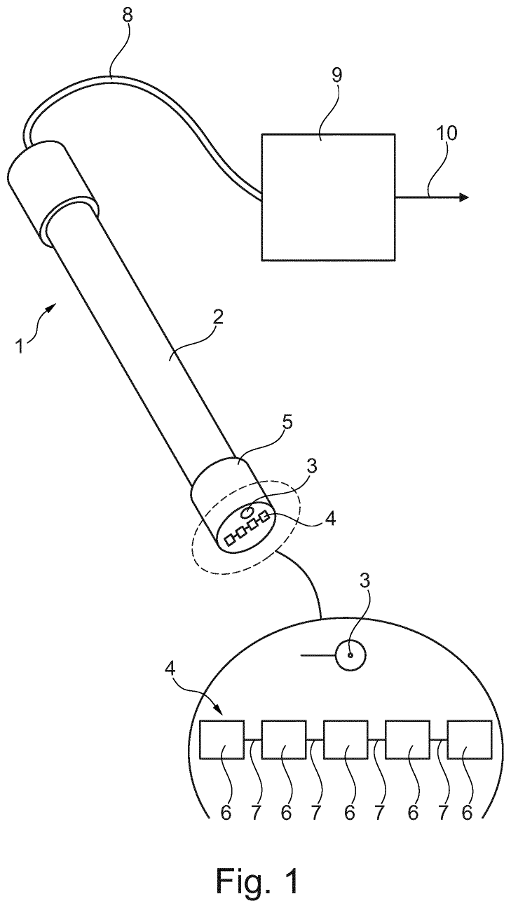

FIG. 1 shows an amperometric sensor 1 as contemplated herein. This sensor 1 has a cylindrical body 2, which surface is the counter electrode surface. Typically the counter electrode surface is titanium and is made large in comparison to the electrodes 3, 4 arranged in the tip 5 of the sensor 1.

The electrode 3 is the working electrode and has typically a silver/silverchloride electrode surface. The reference electrode 4 is composed out of a string of surface parts 6 connected by an electrically conducting track 7.

The electrodes 3, 4 are arranged as layers on a substrate by using wafer production techniques, such that a chip is provided, which is easily mounted in the tip 5 of the sensor 1.

The electrodes 2, 3, 4 are connected via a lead 8 to a control device 9, which has an potentiostat for maintaining the potential of the working electrode 3 at a constant level with respect to the reference electrode 4 by adjusting the current at the counter electrode 2. The control device 9 also has measuring feature, which output a signal 10, which is indicative for the amount of ions in a solution.

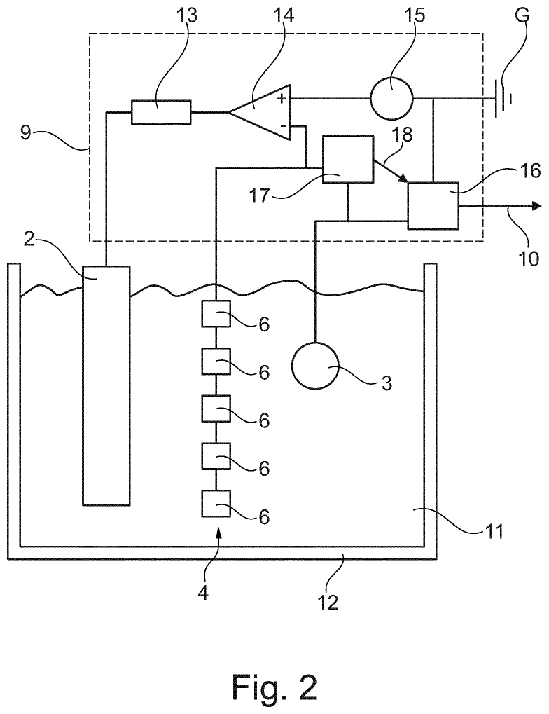

FIG. 2 shows a schematic diagram of a combination as contemplated herein with an amperometric sensor 1 submerged in a solution 11 in a container 12.

The control device 9 has a potentiostat composed out of a resistor 13, an operational amplifier 14 and a power source 15. The potentiostat could also be more complex, depending on the requirements.

The working electrode 3 is connected in the potentiostat to ground G via a current sensor 16, which outputs the signal 10.

To further increase the accuracy of the sensor 1, a resistance sensor 17 is provided, which measures the resistance between the working electrode 3 and the reference electrode 4 and provides a compensation signal 18 with which the output signal 10 is corrected for presence of salts in the solution 11.

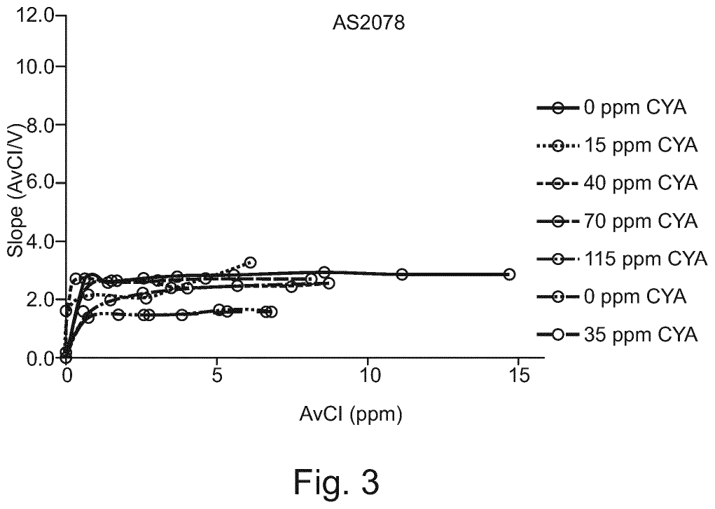

FIG. 3 shows a diagram of the influence of cyanuric acid on the sensitivity of an embodiment of the amperometric sensor as contemplated herein. The Y-axis (Slope (AvCl/V)) presents the sensitivity of the sensor. A higher value corresponds with a reduced sensitivity.

It is clearly shown that independent of the concentration of cyanuric acid (CYA), the amperometric sensor as contemplated herein has a constant sensitivity for a wide range in concentration of free chlorine (AvCl).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing detailed description will provide those skilled in the art with a convenient road map for implementing an exemplary embodiment of the present disclosure. It being understood that various changes may be made in the function and arrangement of elements described in an exemplary embodiment without departing from the scope of the present disclosure as set forth in the appended claims.

What is claimed is:

1. Amperometric sensor for measuring free chlorine, which sensor comprises:
an elongate body with a tip, wherein a circumferential surface of the body constitutes a counter electrode;
a reference electrode having a gold electrode surface arranged on the tip of the elongate body; and
a working electrode having a silver/silverchloride electrode surface arranged on the tip of the elongate body;
wherein
the gold electrode surface is composed out of a string of electrically connected, spaced apart surface parts.

2. Amperometric sensor according to claim 1, wherein the surface parts are identically shaped and dimensioned.

3. Amperometric sensor according to claim 1, wherein the gold electrode surface is provided as a layer arranged on a substrate by wafer production techniques.

4. Amperometric sensor according to claim 1, wherein the counter electrode has a titanium electrode surface.

5. Amperometric sensor according to claim 1, wherein the reference electrode is composed out of a sintered Ag/AgCl powder.

6. Combination of an amperometric sensor according to claim 1 and a potentiostat connected to the counter electrode, the working electrode and the reference electrode for maintaining the potential of the working electrode at a constant level with respect to the reference electrode by adjusting the current at the counter electrode.

7. Combination according to claim 6, wherein the potentiostat comprises a control feature for maintaining the potential of the working electrode at a constant level with respect to the reference electrode, which control feature has a compensating feature, which measures the resistance between electrode surfaces of at least the working electrode and the reference electrode and compensate for the measured resistance.

8. Combination according to claim 6, wherein the potentiostat further comprises a signal generator for adjusting the constant level according to a predefined signal pattern.

9. Amperometric sensor according to claim 2, wherein the surface parts are rectangular.

10. Amperometric sensor according to claim 3, wherein the gold electrode surface is provided as the layer arranged on the substrate by lithographic production techniques.
